(19) United States
(12) Patent Application Publication
(10) Pub. No.: US 2009/0234376 A1
(43) Pub. Date: Sep. 17, 2009

(54) Surgical material applicator

(76) Inventors: Michael Andrew Soltz, New York, NY (US); Robert Soltz, Spring Valley, NY (US); Raymond J. Lanzafame, Rochester, NY (US)

Correspondence Address: Barbara A. Soltz;Conversion Energy Enterprises, #81 Pinebrook Rd., Spring Valley, NY, 10977, US

(21) Appl. No.: 12/378,568

(22) Filed: Feb. 17, 2009

Related U.S. Application Data
(60) Provisional application No. 61/066,071, filed on Feb. 15, 2008.

(51) Int. Cl. A61B 17/03

(52) U.S. Cl. 606/151

(57) ABSTRACT

Consistent with an aspect of the present disclosure, a mesh or other surgical material is wrapped about one or more shafts, and inserted into a body cavity where it is unfurled and placed over a tissue site, such as a hernia. The mesh, for example, is secured to the shaft in order to prevent crimping or crumpling thereof during deployment over the tissue site.

This application claims priority to U.S. Provisional Patent Application Ser. No. 61/066,071 filed on Feb. 15, 2008, which is incorporated by reference herein in its entirety.

### STATEMENT OF RIGHTS TO INVENTIONS MADE UNDER FEDERALLY SPONSORED RESEARCH

This work was supported in part by the National Institute of Diabetes and Digestive and Kidney Diseases grant number 2R44 DK062571. The government has certain rights in the invention.

### BACKGROUND TO THE INVENTION

In many surgical procedures, a surgical material is introduced into a body cavity and placed over a wound or tissue defect. For example, in a hernia repair procedure, a mesh is placed over a hernia in order to repair it. Many different meshes have been developed for this purpose. For example, meshes having different thicknesses and other structural properties are used for different size hernias. Moreover, meshes have been coated with materials to reduce inflammation of adjacent tissues and to minimize adhesions.

In all these repair methods there is a need for an apparatus for quickly introducing and manipulating a prosthetic material while performing open or laparoscopic procedures. Conventional methods, including a variety of mesh applicators, are inefficient, time consuming and unreliable. The mesh is often difficult to attach to the rod and thus may slip about the rod. As a result, the mesh may not unfurl through the opening in the housing. Alternatively, the mesh may crumple within the housing.

### SUMMARY OF THE INVENTION

Consistent with an aspect of the present disclosure a surgical tool is provided that comprises a sheath having an opening. The opening extends along a length of the sheath, the sheath having an inner surface that defines a cavity. The surgical tool also includes a shaft having a surface. The cavity of the sheath is adapted to receive the shaft and a material disposed about the shaft. A plurality of protrusions are also provided that extend from the surface of the shaft. The plurality of protrusions are provided at a plurality of locations along a length of the shaft, and the plurality of protrusions are configured to grip a first portion of the material. Upon rotation of the shaft within the cavity, a second portion of the material is output from the opening for application to a biological tissue.

Consistent with a further aspect of the present disclosure, a surgical tool is provided that comprises a first shaft configured such that a material may be wrapped around the first shaft. The surgical tool also includes a second shaft spaced from the first shaft by a gap. A first member is also provided having first and second recessed portions. The first recessed portion is configured to receive a first end portion of the first shaft, and the second recessed portion is configured to receive a first end portion of the second shaft. In addition, a second member is provided that has first and second recessed portions. The first recessed portion of the second member is configured to receive a second end portion of the first shaft, and the second recessed portion of the second member is configured to receive a second end portion of the second shaft. The first end portion of the first shaft is provided opposite the second end portion of the first shaft, and the first end portion of the second shaft is provided opposite the second end portion of the second shaft. A first portion of the surgical material is inserted through the gap between the two shafts such that the two shafts will grip the first portion of the surgical material. Upon rotation of the two shafts together within the cavity a second portion, of the material is output from the opening for application to a biological tissue.

Additional objects will be set forth in part in the description which follows, or may be learned by practice of the disclosure. The objects and advantages of the will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate several embodiments and together with the description, serve to explain the principles of the disclosure.

### BRIEF DESCRIPTION OF THE DRAWINGS

FIG. 1 is a perspective view of a surgical tool consistent with an aspect of the present disclosure;

FIG. 2 is a perspective view of a sheath consistent with an additional aspect of the present disclosure;

FIG. 3 is perspective view of a shaft consistent with a further aspect of the present disclosure;

FIGS. 4a and 4b show example of the surgical tool consistent with the present disclosure with a mesh retained (FIG. 4a) and a deployed mesh (FIG. 4b);

FIG. 5 shows a partial perspective view of the surgical tool consistent with the present disclosure;

FIG. 6-10 show perspective views of examples of a shaft consistent with the present disclosure;

FIG. 11 illustrates an alternative embodiment of a portion of a surgical tool consistent with the present disclosure;

FIG. 11a illustrates a cross-sectional view of the surgical tool of FIG. 11 with the surgical material wound onto the shaft;

FIG. 12 illustrates an example of an application of a surgical tool consistent with the present disclosure; and

FIGS. 13a-13d illustrate different views of tips of a shaft consistent with the present disclosure.

### DESCRIPTION OF THE EMBODIMENTS

Consistent with an aspect of the present disclosure, surgical material is contained within the sheath while it is introduced into the abdominal cavity, thereby preventing a premature discharge of the mesh before it is properly positioned over the defect. As a result, material introduction is low risk, quick, inexpensive and reproducible. Specifically, the surgical material is prevented from folding over on itself and will provide the necessary control to achieve the desired registration and orientation of the surgical material within the abdominal cavity. Commonly used composite materials including relatively thin meshes that are structurally weak as well as thick, rigid materials can be reliably positioned so that the coated or adhesion barrier side of the material will typically be placed toward the bowel while the uncoated side will face the abdominal wall. In this manner, adhesion formation and viscus erosion can be prevented. Furthermore thick, large sheets of a surgical material can be compressed onto the apparatus, eliminating complications associated with use of large-bore cannulas. In addition, components of the apparatus protect the surgical material from damage as it is transported through a hard trocar material. Alternatively, the apparatus can be inserted directly into a trocar opening once the trocar is removed. Benefits to this disclosure include a shaft that is populated with spines, bristles or rounded features that gently grasp any surgical material preventing it from slipping on the shaft and faciliting tightly winding it onto the shaft. As a result large-sized, stiff materials as well as delicate thin materials can be compressed within this device for easy application to the defect site. Another benefit is the outer sheath of the device that protects the surgical material that is wound onto the shaft preventing damage to the material when it is inserted into a trocar in order to execute laparoscopic procedures. Another benefit is a specially designed tunnelling member that facilities insertion into the trocar as well as an aid in positioning the surgical material at the defect site once the material has been released from the shaft. The combination of controlled release of the material from the device with placement assistance of the distal tip, facilitates the correct registration of composite materials to ensure that the coated or postoperative adhesion barrier surface is positioned toward abdominal tissues. Another benefit is a seal within the device to prevent escape of suffalation gas during a laparoscopic procedure.

Reference will now be made in detail to the present exemplary embodiments, examples of which are illustrated in the accompanying drawings. Wherever possible, the same reference numbers will be used throughout the drawings to refer to the same or like parts.

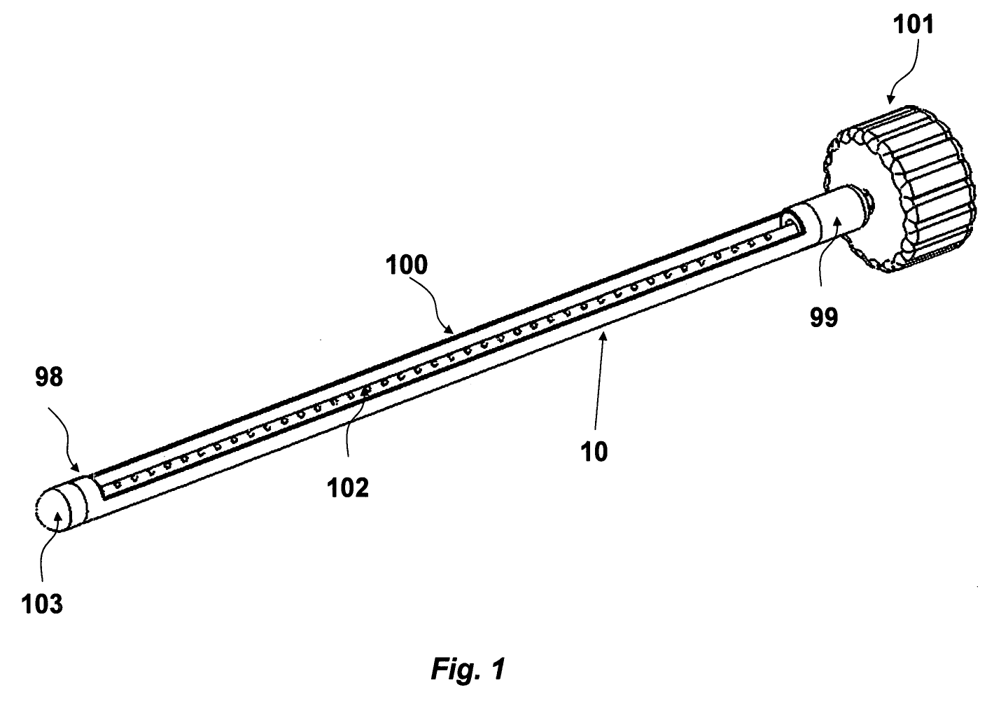

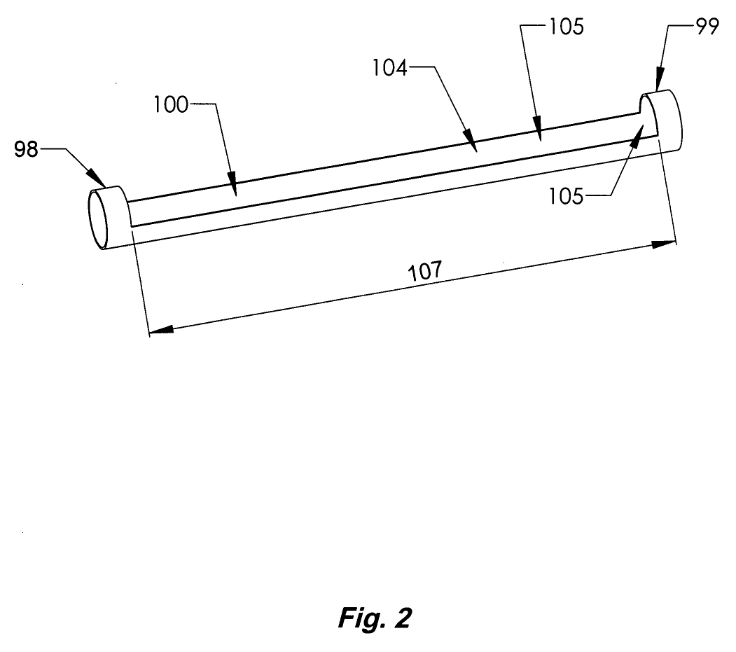

FIG. 1 illustrates a surgical tool 10 consistent with an aspect of the present disclosure. Surgical tool 10 includes a shaft 102 provided in a sheath 100. A tip 103 may be provided at a distal or first end portion 98 of sheath 100 and a handle or knob 101 may be provided at a proximal or second end portion 99 of sheath 100. As shown in greater detail in FIG. 2, sheath 100 has an inner surface 105 that defines a cavity 106. An opening 104 is provided in sheath 100 that extends along a length 107 of sheath 100.

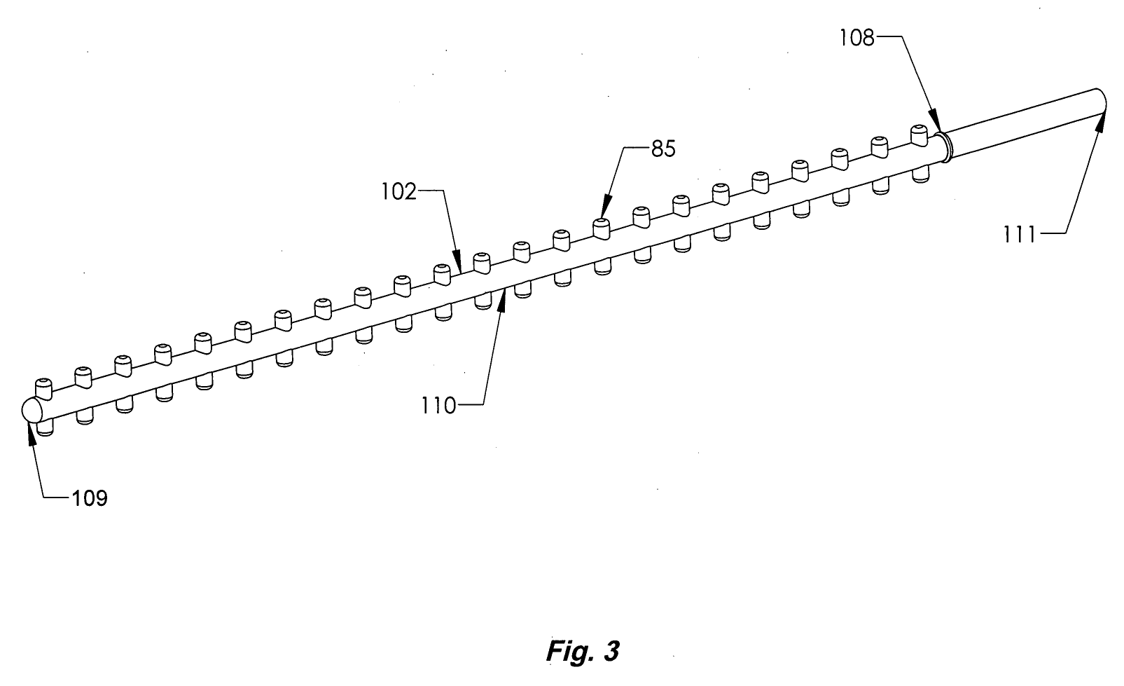

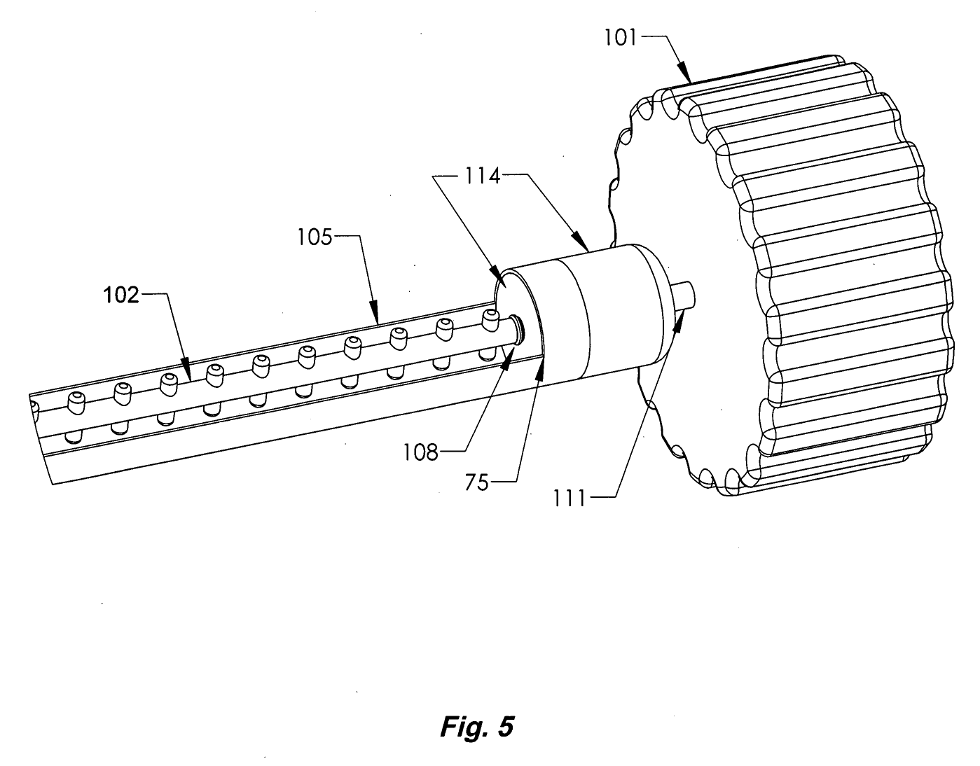

FIG. 3 illustrates an exemplary shaft 102 in greater detail. Shaft 102 includes a plurality of protrusions 110 that are provided at a plurality of locations that extend from surface 85 along the length of shaft 102 from distal end 109 to proximal end 111. Shaft 102 may also include a raised diameter portion 108, which may include an O-ring, for example, near proximal end 111. Raised diameter portion 108 will be discussed in greater detail below with reference to FIG. 5.

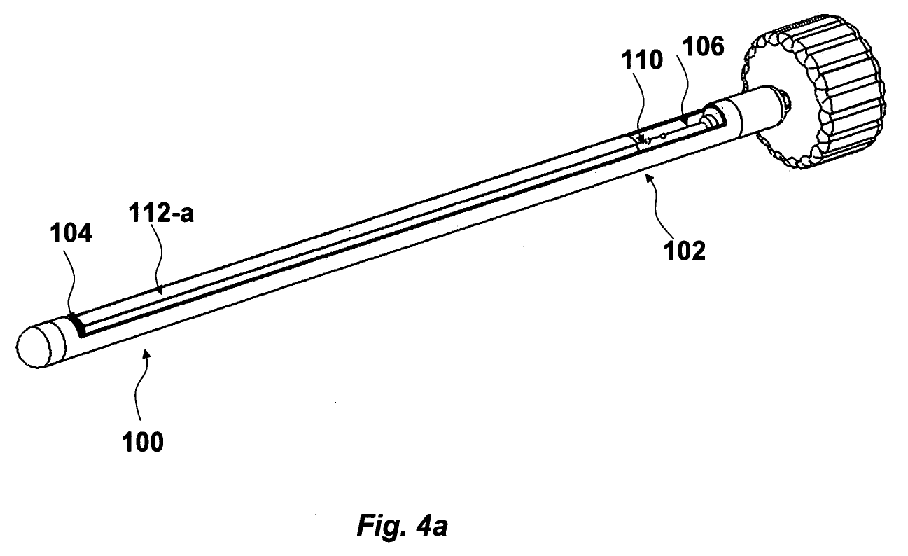

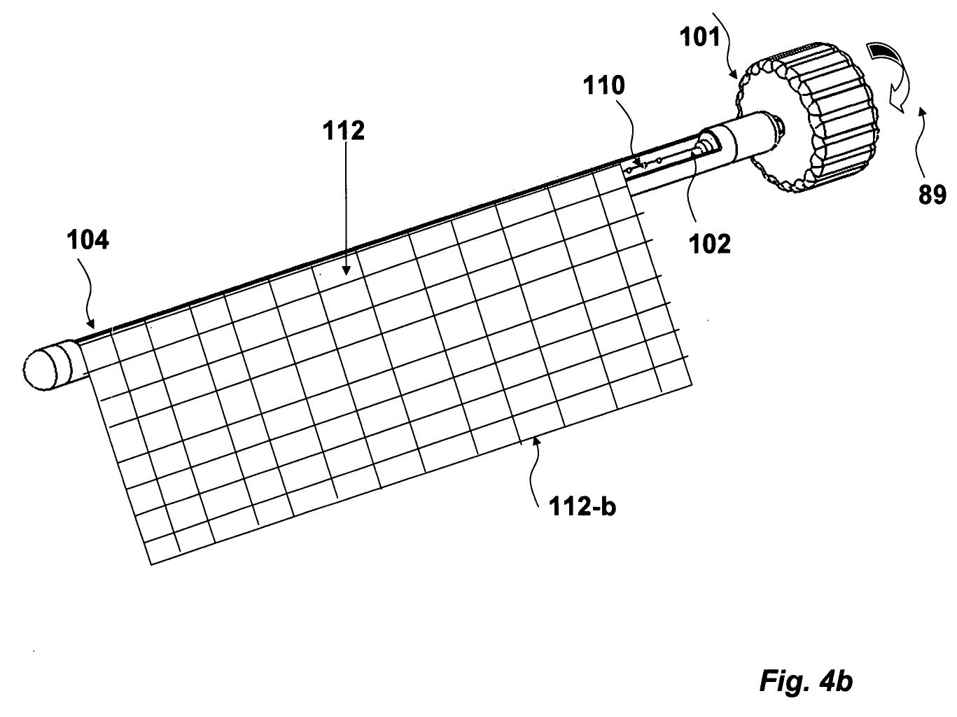

The operation of surgical tool 10 will next be described with reference to FIGS. 4a and 4b. In FIG. 4a, at least a first portion 112-a of material, such as a mesh 112 is wrapped around shaft 102 within cavity 106 of sheath 100. In FIG. 4b, handle 101 is rotated in a direction indicated by arrow 89 by a user, for example, such that at least a second portion 112-b of mesh 112 is unfurled and supplied from opening 104. Preferably, protrusions 110 along shaft 102 are configured to grip mesh 112, such that mesh 112 may be reliably unfurled from shaft 102 without slippage. As a result, mesh 112 may be properly positioned over a biological tissue or surgical site, such as a hernia. Moreover, the height and width of protrusions 110 are preferably selected so that a variety of meshes having a wide range of thicknesses may be effectively engaged and suitably wound and unwound by shaft 102.

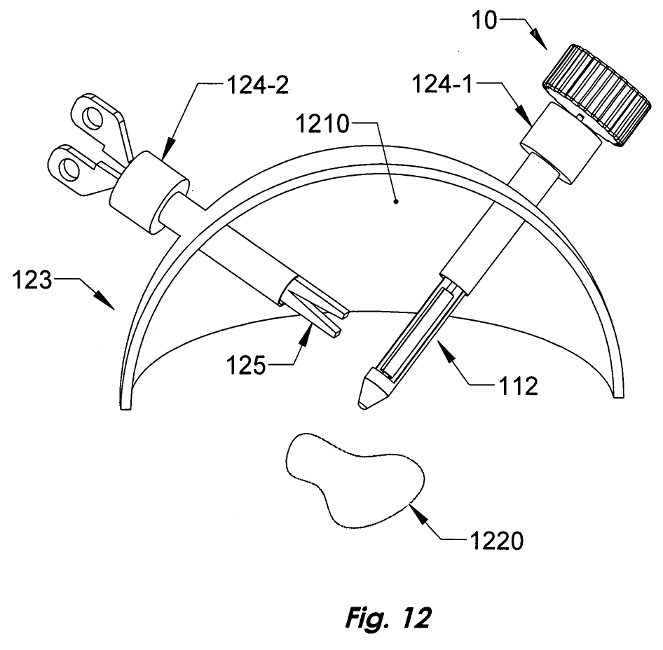

FIG. 5 illustrates proximal end 111 of shaft 102 in greater detail. As noted above, a raised diameter portion 108 may be provided near proximal end 111 in order to form a seal against a portion 75 of space seal 114. Spacer seal 114 forms a seal between the shaft and the shealth inner surface 105. Accordingly, gasses or fluids released from a body cavity when surgical tool 10 is inserted therein may be contained. An additional spacer seal 114, which may include a glass, may be provided to provide a proper fit of surgical tool 10 within a trocar (discussed in greater detail below with respect to FIG. 12). Preferably, seals 108 and 114 have a central opening to thereby facilitate a connection or attachment between handle 101 and shaft 102 to facilitate rotation of shaft 102 by turning handle 101 either manually or with a motor, for example.

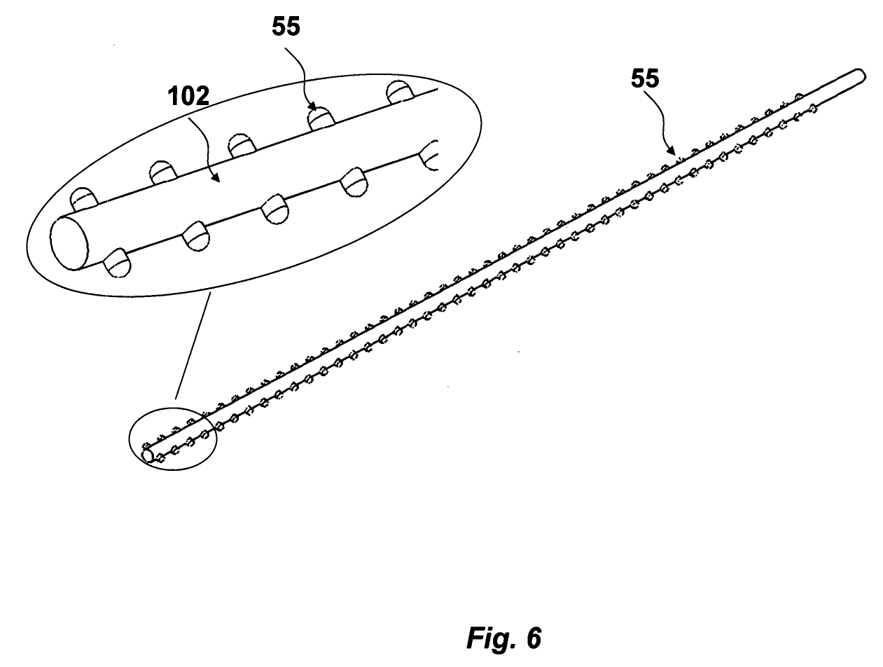

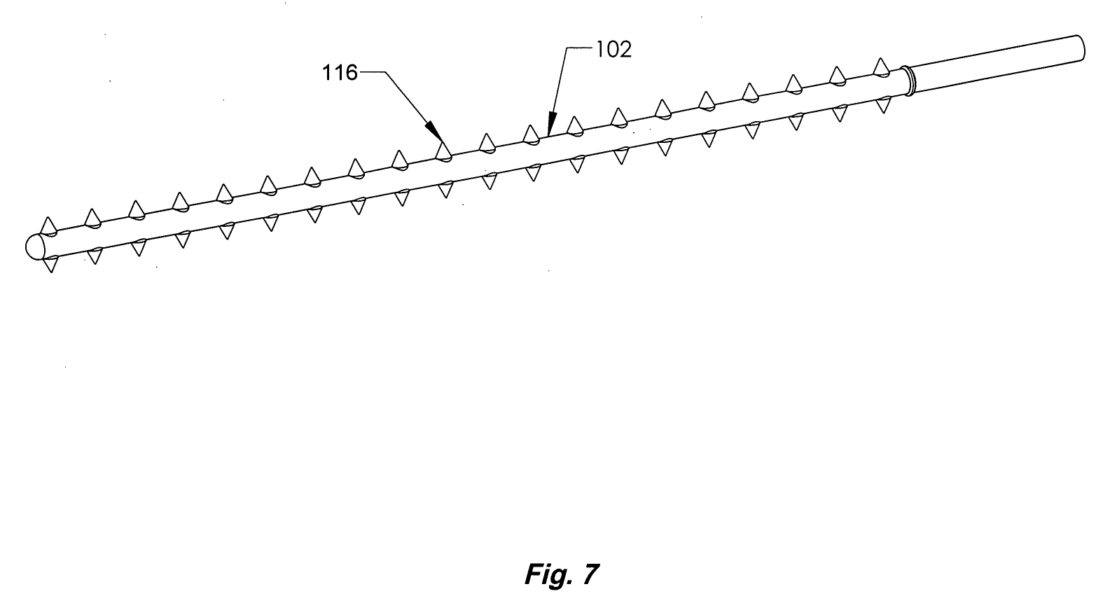

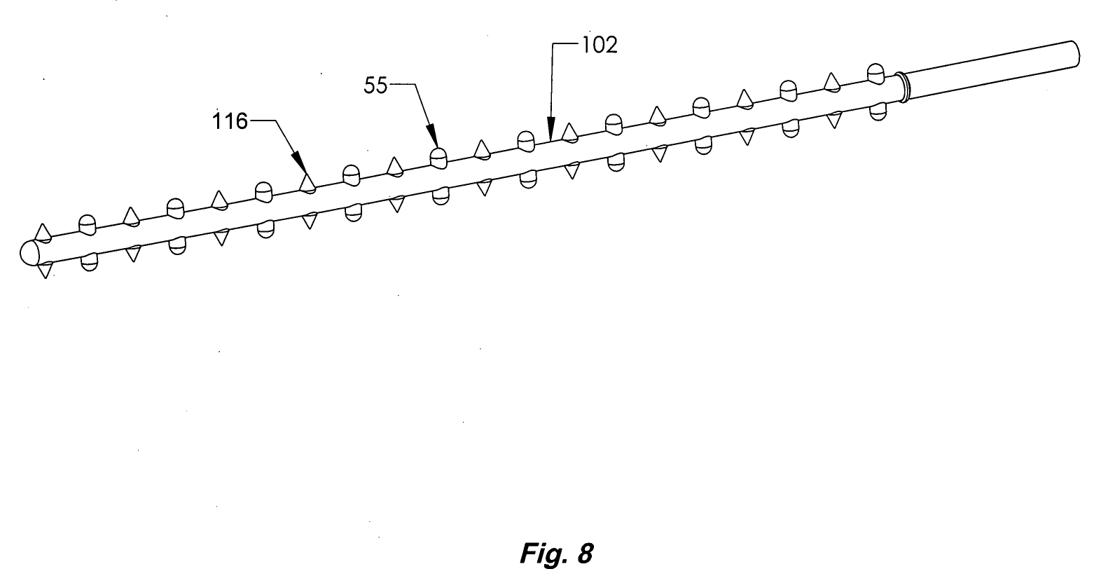

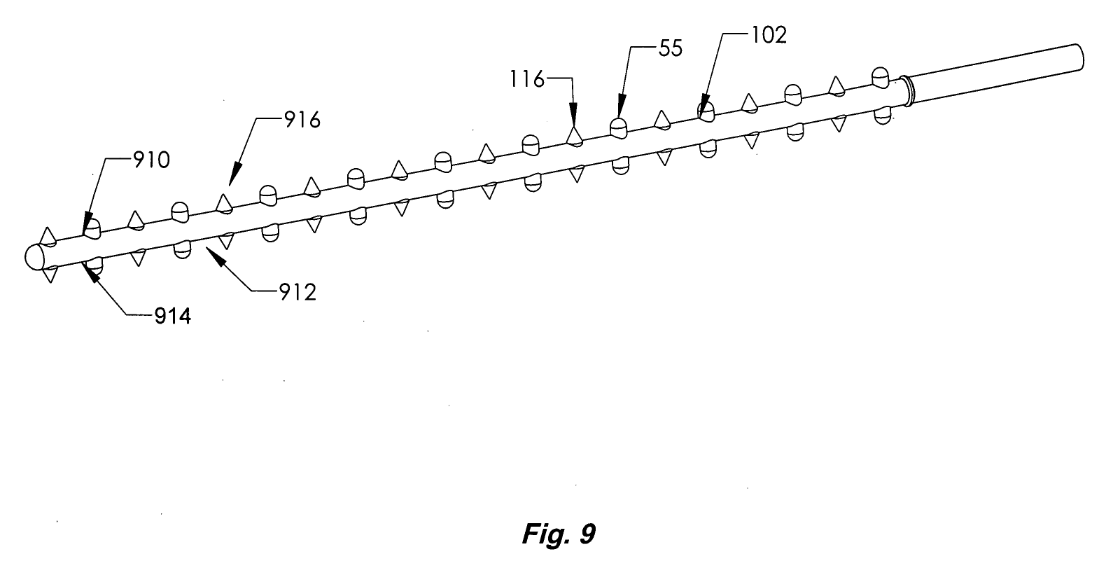

FIG. 6 shows shaft 102 in greater detail. In this example, a plurality of protrusions 55, having a rounded shape, are provided on opposite sides of shaft 102. In FIG. 7, a plurality of protrusions 116 or spikes, having a conical shape, are provided on opposite sides of shaft 102. In an additional example shown in FIG. 8, rounded (55) and conical (116) protrusions are provided at alternating positions along shaft 102. In FIG. 9, a further example, conical-shaped protrusions 116 are provided on opposing first (910) and second (912) sides along the length of shaft 102, and rounded protrusions 55 are provided on opposing first (914) and second (916) sides of shaft 102. Depending upon the thickness, rigidity, and other physical properties of the mesh or other material to be deployed with surgical tool 10, different protrusion stapes provided at different locations along the length of shaft 102 may facilitate better gripping of the mesh or other surgical material.

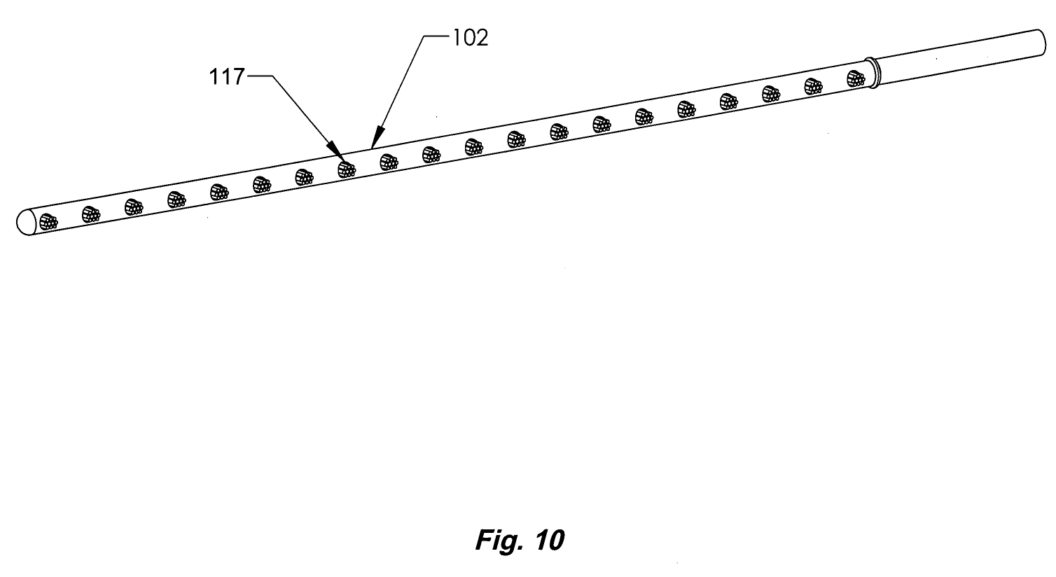

FIG. 10 illustrates another example of shaft 102. Here, shaft 102 includes a plurality of bristles, arranged in clumps 117 spaced along the length of the shaft. Bristles may be used to grip the mesh or other surgical material in a manner similar to that discussed above in connection with protrusions 55 and 116.

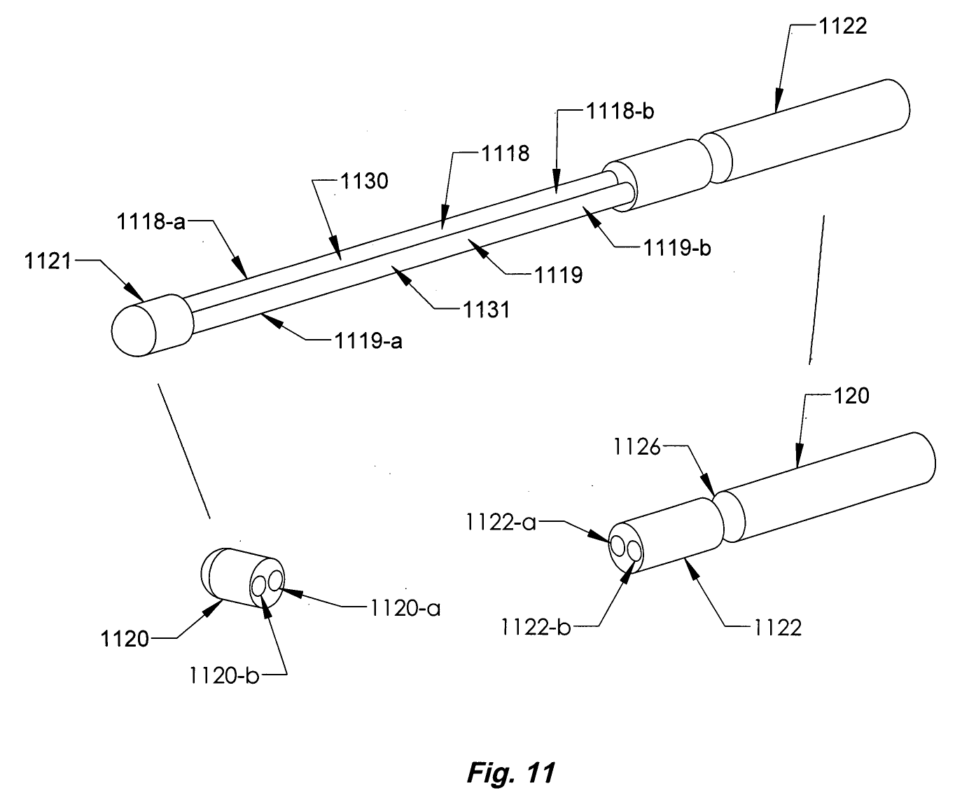

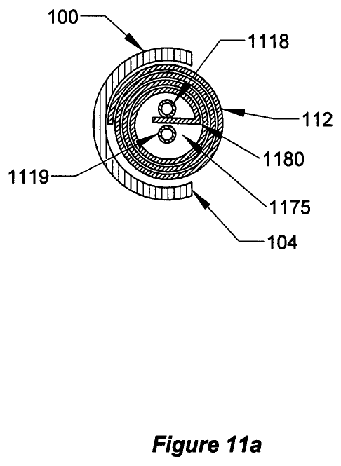

FIG. 11 illustrates an example of a dual-shaft configuration 1100 consistent with a further aspect of the present disclosure. Configuration 1100 includes first 1118 and second 1119 shafts that are spaced from one another by a gap. At distal end 1121, first ends 1118-a and 1119-a of the first and second shafts, respectively, are provided in corresponding recessed portions 1120-a and 1120-b in distal member 1120. At proximal end member 1122, second ends 1118-b and 1119-b of shafts 1118 and 1119, respectively, are provided in recessed portions 1122-a and 1122-b. Portion 120 is also provided which is may extend in and connect to handle 101 (not shown in FIG. 11) to facilitate rotation of shafts 1118 and 1119. In addition, recessed portion 1125 may accommodate an O-ring or other seal to thereby realize raised diameter portion 108.

FIG. 11a shows a cross-sectional view AA from FIG. 11. In operation, a first portion 1175 of mesh 112, for example, is secured in gap 1180 between shafts 1118 and 1119, and then wrapped around both shafts, as shown in the cross-sectional view of surgical tool 10 and shown in FIG. 11a. Preferably, portion 120 (extension or portion of handle 101 not shown) is turned to facilitate rotation of shafts 1118 and 1119 and unfurling of at least a second portion of mesh 112 through opening 104 in sheath 100. Gap 1180 is typically set to approximate the thickness of the mesh to retain portion 1175 of mesh 112 and avoid crimping or crumpling of the mesh while it is unfurled. Although the shafts 1118 and 1119 and corresponding recessed portions 1120-a and 1120-b of member 1120 are shown having the same diameter, it is understood that each may have a different cross-sectional area when taken transverse to the axes 1130 and 1131 of shafts 1118 and 1119, respectively and shown in FIG. 11. In the example shown in FIGS. 11 and 11a, the material is gripped by gap 1180 between shafts 1118 and 1119, instead of or in addition to protrusions 110 discussed above.

FIG. 12 illustrates an exemplary application of surgical tool 10 consistent with an additional aspect of the present disclosure. In the example shown in FIG. 12, surgical tool 10, including mesh 112, is provided within first trocar 124-1 and inserted into abdominal wall 123. A second trocar 124-2, including a grabber mechanism 125, may also be inserted into abdominal wall 123. Grabber mechanism 125, which may include a pair of tongs 1210, for example, may be used to grab mesh 112 and unfurl or extend, as well as position, it over a hernia or other surgical site 1220. Typically, sheath 100 protects mesh 112 during insertion into trocar 124-1.

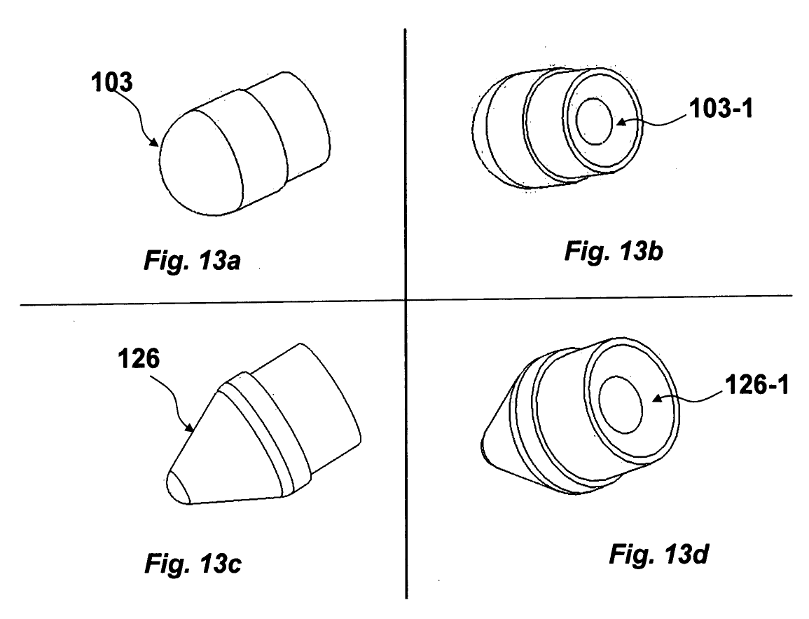

FIGS. 13a and 13b illustrate examples of tunneling members or tips that may be placed at the distal end 98 of sheath 100. FIGS. 13a and 13b illustrate front and rear views, respectively, of tunneling member 103 having a rounded shape. FIG. 13b, in particular, shows an opening 103-1 for receiving distal end 109 of shaft 102. FIGS. 13c and 13d illustrate front and rear views, respectively, of a tunneling member 126 having a conical shape, and FIG. 13d shows opening 126-1 for receiving the distal end 109 of shaft 102. In each of FIGS. 13a-13d, the tips have a rounded (in the case of tip 103) or conical (in the case of tip 126) that protrudes in a direction away from proximal end 111 of shaft 102. For the two shaft configuration shown in FIG. 11, the round and conical shaped tips are similar to those shown in FIGS. 13a and 13c but the two shaft configuration requires two openings as shown in FIG. 11 view 1120 (1120-a and 1120-b).

Tips or tunneling members 103 and 126 permit easy movement of surgical tool 10 without snagging or damaging underlying biological tissue. Accordingly, surgical tool 10 may be readily positioned over a desired location, and the mesh or other surgical material may be properly deployed in the manner described above.

As noted above and consistent with aspects of the present disclosure, protrusions provided along the shaft are provided to facilitate gripping of the mesh, thereby facilitating crimp-free deployment and accurate positioning of the mesh over the surgical site, such as hernia, preventing damage or irritation to adjacent tissues and possible reducing the formation of postoperative adhesions. Alternatively, the mesh may be gripped by securing a portion thereof in a gap between two closely spaced shafts.

Other embodiments will be apparent to those skilled in the art from consideration of the specification and practice of the invention disclosed herein. For example, in each of the above-noted examples, the shafts may be made of a suitable plastic or metal. It is intended that the specification and examples be considered as exemplary only, with a true scope and spirit of the invention being indicated by the following claims.

What is claimed is:

1. A surgical tool comprising:
a sheath having an opening, the opening extending along a length of the sheath, the sheath having an inner surface defining a cavity; and
a shaft, the cavity of the sheath being adapted to receive the shaft and a material disposed about the shaft, the shaft having surface; and
a plurality of protrusions extending from the surface of the shaft, the plurality of protrusions being provided at a plurality of locations along a length of the shaft, the plurality of protrusions being configured to grip a first portion of the material, wherein, upon rotation of the shaft within the cavity, a second portion of the material is output from the opening for application to a biological tissue.

2. A surgical tool in accordance with claim 1, wherein the each of the plurality of protrusions has a rounded shape.

3. A surgical tool in accordance with claim 1, wherein each of the plurality of protrusions has a conical shape.

4. A surgical tool in accordance with claim 1, further including a handle attached to an end portion of the shaft, the handle being configured to be gripped by a user to facilitate said rotation of the shaft with the cavity.

5. A surgical tool in accordance with claim 1, wherein the shaft includes metal.

6. A surgical tool in accordance with claim 1, wherein the shaft includes plastic.

7. A surgical tool in accordance with 4, wherein the end portion is a first end portion, the shaft having a second end portion provided opposite the first end portion, the second portion having a rounded shape that protrudes in a direction away from the first end portion.

8. A surgical tool in accordance with 4, wherein the end portion is a first end portion, the shaft having a second end portion provided opposite the first end portion, the second portion having a conical shape that protrudes in a direction away from the first end portion.

9. A surgical tool in accordance with claim 1, wherein the material constitutes a mesh.

10. A surgical tool in accordance with claim 1, wherein first ones of the plurality of protrusions are linearly arranged in a first row provided on a first side of the shaft and second ones of the plurality of protrusions are linearly arranged in a second row provided on a second side of the shaft.

11. A surgical tool in accordance with claim 10, wherein the first side of the shaft is opposite the second side of the shaft.

12. A surgical tool in accordance with claim 1, further including a trocar, the trocar having an opening configured to receive at least a portion of the sheath and a portion of the shaft.

13. A surgical tool comprising:
a first shaft;
a second shaft spaced from the first shaft by a gap, the gap being configured to receive a first portion of a material;
a first member having first and second recessed portions, the first recessed portion configured to receive a first end portion of the first shaft, and the second recessed portion configured to receive a first end portion of the second shaft;
a second member having first and second recessed portions, the first recessed portion of the second member being configured to receive a second end portion of the first shaft, and the second recessed portion of the second member being configured to receive a second end portion of the second shaft, the first end portion of the first shaft being opposite the second end portion of the first shaft, and the first end portion of the second shaft being opposite the second end portion of the second shaft; and
a sheath having an opening, the opening extending along a length of the sheath, the sheath having an inner surface defining a cavity, the first and second shafts being provided in the cavity,
wherein, upon rotation of the first shaft, a second portion of the material is provided through the gap between the first and second shafts for application to a biological tissue.

14. A surgical tool in accordance with claim 13, further including a handle attached to an end portion of the shaft, the handle being configured to be gripped by a user to facilitate said rotation of the first and second shafts within the cavity.

15. A surgical tool in accordance with claim 13, wherein the shaft includes metal.

16. A surgical tool in accordance with claim 13, wherein the shaft includes plastic.

17. A surgical tool in accordance with 14, wherein the end portion is a first end portion, the shaft having a second end portion provided opposite the first end portion, the second portion having a rounded shape that protrudes in a direction away from the first end portion.

18. A surgical tool in accordance with 14, wherein the end portion is a first end portion, the shaft having a second end portion provided opposite the first end portion, the second portion having a conical shape that protrudes in a direction away from the first end portion.

19. A surgical tool in accordance with claim 13, wherein the material constitutes a mesh.

20. A surgical tool in accordance with claim 13, further including a trocar, the trocar having an opening configured to receive at least a portion of the sheath and a portion of the shaft.

21. A surgical tool comprising:
a sheath having an opening, the opening extending along a length of the sheath, the sheath having an inner surface defining a cavity; and
a shaft, the cavity of the sheath being adapted to receive the shaft and a material disposed about the shaft, the shaft having surface; and
a plurality of bristles extending from the surface of the shaft, the plurality of bristles being configured to grip a first portion of the material, wherein, upon rotation of the shaft within the cavity, the material is output from the opening for application to a biological tissue.

22. A surgical tool in accordance with claim 21, further including a handle attached to an end portion of the shaft, the handle being configured to be gripped by a user to facilitate said rotation of the shaft with the cavity.

23. A surgical tool in accordance with claim 21, wherein the shaft includes metal.

24. A surgical tool in accordance with claim 21, wherein the shaft includes plastic.

25. A surgical tool in accordance with 22, wherein the end portion is a first end portion, the shaft having a second end portion provided opposite the first end portion, the second portion having a rounded shape that protrudes in a direction away from the first end portion.

26. A surgical tool in accordance with 22, wherein the end portion is a first end portion, the shaft having a second end portion provided opposite the first end portion, the second portion having a conical shape that protrudes in a direction away from the first end portion.

27. A surgical tool in accordance with claim 21, wherein the material constitutes a mesh.

28. A surgical tool in accordance with claim 21, wherein first ones of the plurality of bristles are linearly arranged in a first row provided on a first side of the shaft and second ones of the plurality of bristles are linearly arranged in a second row provided on a second side of the shaft.

29. A surgical tool in accordance with claim 28, wherein the first side of the shaft is opposite the second side of the shaft.

30. A surgical tool in accordance with claim 21, further including a trocar, the trocar having an opening configured to receive at least a portion of the sheath and a portion of the shaft.

31. A surgical tool in accordance with claim 13, wherein the first shaft extends along a first longitudinal axis and the second shaft extends along a second longitudinal axis, the first shaft having a first cross-sectional area which is transverse to the first longitudinal axis and the second shaft having a second cross-sectional area which is transverse to the second longitudinal axis, the first cross-sectional area being greater than the second cross-sectional area.
